(19) United States
(12) Patent Application Publication
(10) Pub. No.: US 2020/0113802 A1
(43) Pub. Date: Apr. 16, 2020

(54) STYLING COMPOSITION HAVING DYEING PROPERTIES

(71) Applicant: Henkel AG & Co. KGaA, Duesseldorf (DE)

(72) Inventors: MARCUS NOLL, Quickborn (DE); THORSTEN KNAPPE, Schenefeld (DE); MARIE BORSON, Hamburg (DE)

(73) Assignee: Henkel AG & Co. KGaA, Duesseldorf (DE)

(21) Appl. No.: 16/597,165

(22) Filed: Oct. 9, 2019

(30) Foreign Application Priority Data
Oct. 11, 2018 (DE) 10 2018 217 367.2

(51) Int. Cl. A61K 8/73; A61K 8/92; A61K 8/31; A61K 8/81; A61Q 5/06

(52) U.S. Cl. CPC A61K 8/732; A61K 8/925; A61K 8/31; A61K 8/8176; A61K 2800/43; A61Q 5/065; A61K 2800/30; A61K 2800/62; A61K 8/8182

(57) ABSTRACT

A cosmetic composition for the temporary deformation of keratinic fibers comprises, based on its total weight
a) from about 1 to about 10% by weight of starch particles comprising, based on the weight of the starch particles,
   a1) from about 70 to about 99% by weight of starch, and
   a2) from about 1 to about 30% by weight of pigment, and
b) from about 0.5 to about 50% by weight of a firming compound selected from the group comprising waxes, synthetic polymers and mixtures thereof.

### CROSS-REFERENCE TO RELATED APPLICATION

This application claims priority to German Patent Application No. 10 2018 217 367.2, filed Oct. 11, 2018, which is incorporated herein by reference in its entirety.

### TECHNICAL FIELD

The application relates to a cosmetic composition for the temporary deformation of keratinic fibers comprising a starch, a pigment and a firming compound.

### BACKGROUND

The production of melanin, a pigment responsible for hair coloration, can begin to decline by the end of 20. The result: the hair gradually loses its color until finally all hair on the head is grayed.

The days when only women tinted and bleached their hair to cover the first gray strands are long gone. According to surveys by market research institutes, men dye their gray hair, especially if they hope to thereby gain advantage in their job. Many men, however, shy away from using permanent hair dye.

Products specially developed for men's hair, so-called repigmentation creams, can be applied easily. They work quickly and safely. They are used like a shampoo. The hair is filled with natural pigments with each hair wash.

However, there is still a need for cosmetic compositions that in particular can be integrated in the daily cleaning, care and/or styling routine, without requiring the use of an additional agent/productAccordingly, objects, desirable features and characteristics of the styling composition contemplated herein will become apparent from the subsequent summary and detailed description, and the appended claims, taken in conjunction with this background.

### BRIEF SUMMARY

In accordance with an exemplary embodiment, a cosmetic composition for the temporary deformation of keratinic fibers is provided. The cosmetic composition comprises, based on its total weight,
a) from about 1 to about 10% by weight of starch particles comprising, based on the weight of the starch particles,
   a1) from about 70 to about 99% by weight of starch and
   a2) from about 1 to about 30% by weight of pigment,
b) from about 0.5 to about 50% by weight of a firming compound selected from the group comprising waxes, synthetic polymers and mixtures thereof.

In accordance with another exemplary embodiment, a cosmetic composition having dyeing properties is provided. The cosmetic composition comprises:
a) starch particles comprising a starch core coated with a pigment coating; and
b) a firming compound selected from the group comprising waxes, synthetic polymers and mixtures thereof.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Cosmetic compositions for the temporary deformation of keratinic fibers (hereinafter also referred to as styling agents or styling compositions) are used to construct a hairstyle and then give it a long-lasting hold. Styling compositions are usually available in the form of paints, foams, waxes or gels.

The term keratinic fibers as contemplated herein comprises furs, wool and feathers, but in particular human hair such as head hair and/or whiskers.

A first essential ingredient of the cosmetic compositions contemplated herein is the starch particle, which in turn, in addition to starch, further comprises pigment.

“Particles” are understood to mean particulate solids at about 25° C. and about 1013 mbar. It is preferred that the starch particles have an average particle diameter of from about 5 to about 100 μm, in particular of from about 10 to about 75 μm.

For the applicability and cosmetic effect of the cosmetic composition, it has proven to be advantageous to limit the proportion by weight of the starch particles in the total weight of the cosmetic composition to values of from about 1.5 to about 8% by weight and in particular from about 2 to about 6% by weight. Corresponding compositions are preferred.

In addition to the proportion by weight of starch particles in the total weight of the cosmetic composition, the composition of the starch particles themselves has proven to be relevant for the cosmetic effect.

The starch particles comprise starch and pigment as their two essential ingredients.

Starch is a reserve carbohydrate, which is stored by many plants in the form of starch grains (granules), which are usually from about 1 to about 200 μm in size, in various parts of plants, for example, in tubers or roots, grain seeds, fruits and in the marrow. Starch belongs to the family of homoglycans and is a polycondensation product of D-glucose. In this case, starch includes three structurally different polymers of d-glucopyranose, namely amylose, amylo pectin and a so-called intermediate fraction.

A preferably usable starch compound is selected from at least one, optionally modified, polycondensation product of D-glucose obtained from starches of potatoes, corn, rice, peas, acorns, chestnuts, barley, wheat, bananas, sago, millet, sorghum, oats, rye, beans, batata, maranta or cassava. More preferably, the cosmetic composition contains at least one starch compound which is tapioca starch, potato starch, corn starch or rice starch. Mixtures of the aforementioned starch compounds are also contemplated herein. Most preferably, the starch compound is rice starch.

Particularly advantageous cosmetic compositions contain starch particles, which in turn, based on their total weight, contain from about 75 to about 96% by weight of starch, in particular from about 80 to about 94% by weight of starch, very particularly preferably rice starch.

The starch particles contained in the cosmetic compositions further contain pigment in addition to the starch. With respect to the styling performance of the cosmetic composition and its optical-cosmetic effect, it has proved to be advantageous when the starch particles, based on their total weight, contain from about 1 to about 30% by weight of pigment, preferably from about 5 to about 25% by weight of pigment. Corresponding starch particles are therefore preferred.

The pigment used is preferably water-insoluble. In a preferred embodiment, the pigment is selected from the group including mineral pigments, metal pigments and pearlescent pigments.

Preference is given to the use of inorganic pigments. These pigments include pigments that are naturally occurring, obtainable by mechanical treatment, and synthetic pigments.

Mineral pigments or natural inorganic pigments predominantly contain sulfides and oxides. Preferred natural pigments are ocher (Fe(OOH); Pigment Yellow 43), calcined Siena (Fe₂O₃; Pigment Red 102), umbra (Fe₂O₃.xMnO₂; Pigment Brown 7:x), cinnabar (β-HgS, PR 106), lapis lazuli (ultramarine, Na₆Al₆Si₆O₂₄.Na₂Sₙ; Pigment Blue 29), azurite (basic copper carbonate Cu₃[OH/CO₃]₂; PB 30), green earth (FeO-containing silicate, Pigment Green 23), malachite (Cu₂[(OH)₂, CO₃]) and carbon black (carbon (graphite), Pigment Black 9).

Powdery metals or metal alloys are referred to as metal pigments or metallic effect pigments. Particularly advantageous are aluminum pigments (aluminum bronze) and brass pigments (so-called gold bronzes), that is, copper/aluminum alloys or copper/zinc alloys, wherein the term “bronze” in this context is not viewed in the narrow chemical sense.
Exemplary metal pigments include:
aluminum bronzes (Al, silver color);
gold bronzes (Cu, Cu—Al or Cu—Zn alloy; natural shades),
(Cu; color copper), (90% Cu-10% Zn; pale gold), (85% Cu-15% Zn; rich pale gold), (70% Cu-30% Zn; rich gold);
silver bronzes (Cu—Zn—Ni; silver)
fire-colored bronzes (oxidized Cu—Zn; tinting colors such as citron, fire red, sea green); and
patent bronzes (Cu—Zn (Ni)+dyes; various colors).

Pearlescent pigments are luster pigments that include several layers of different refractive indices. Pearlescent pigments that are preferred as contemplated herein are magnesium stearate, zinc stearate and lithium stearate or ethylene glycol distearate or polyethylene terephthalate and pearlescent pigments that consist substantially of mica, titanium dioxide (titanium dioxide mica), bismuth oxychloride or guanine, and moreover can be coated with colored oxide layers (for example, iron oxides or chromium oxides).

Pearlescent pigments based on mica and on mica/metal oxide are particularly preferred pearlescent pigments in this case. Mica belongs to the layered silicates. Important representatives of these silicates are muscovite, phlogopite, paragonite, biotite, lepidolite and margarite. To produce the pearlescent pigments in compounds with metal oxides, the mica, predominantly muscovite or phlogopite, is coated with a metal oxide. Suitable metal oxides include TiO₂, Cr₂O₃ and Fe₂O₃. Interference pigments and color luster pigments are obtained as pearlescent pigments preferred as contemplated herein by appropriate coating. These pearlescent pigment types also have color effects in addition to a glittering optical effect. Furthermore, the usable pearlescent pigments can additionally contain a color pigment which does not derive from a metal oxide.

Very particularly preferred pearlescent pigments are marketed by Merck under the trade names Colorona®, including the pigments Colorona® red-brown (from about 47-57% by weight Muscovite mica (KH₂(AlSiO₄)₃), from about 43-50% by weight Fe₂O₃ (INCI: Iron Oxides CI 77491), <3% by weight TiO₂ (INCI: (Titanium Dioxide CI 77891)), Colorona® Blackstar Blue (from about 39-47% by weight Muscovite mica (KH₂(AlSiO₄)₃), from about 53-61% by weight Fe₃O₄ (INCI: Iron oxides CI 77499)), Colorona® Siena Fine (from about 35-45% by weight Muscovite mica (KH₂(AlSiO₄)₃), from about 55-65% by weight Fe₂O₃ (INCI: Iron oxides CI 77491)), Colorona® Aborigine Amber weight (from about 50-62% by weight Muscovite mica (KH₂(AlSiO₄)₃), from about 36-44% by weight Fe₃O₄ (INCI: Iron Oxides CI 77499), from about 2-6% by weight TiO₂ (INCI: Titanium Dioxide CI 77891)), Colorona® Patagonian Purple (from about 42-54% by weight Muscovite mica (KH₂(AlSiO₄)₃), from about 26-32% by weight Fe₂O₃ (INCI: Iron Oxides CI 77491), from about 18-22% by weight TiO₂ (INCI: Titanium Dioxide CI 77891), from about 2-4% by weight Prussian Blue (INCI: Ferric Ferrocyanide CI 77510)), Colorona® Chameleon (from about 40-50% by weight Muscovite mica (KH₂(AlSiO₄)₃), from about 50-60% by weight Fe₂O₃ (INCI: Iron oxides CI 77491)), and Silk® Mica (>98% by weight Muscovite mica (KH₂(AlSiO₄)₃)).

Synthetic inorganic pigments are obtained in particular by chemical and/or physical conversion (decomposition, precipitation, annealing). These include in particular:
white pigments (titanium dioxide (TiO₂), Pigment White PW 6; zinc sulfide (ZnS), PW 7; zinc oxide (ZnO), PW 4; antimony white (Sb₂O₃), PW 11; lithopone (ZnS/BaSO₄), PW 5; lead white (2PbCO₃.Pb(OH)₂), PW 1),
subordinate white fillers (calcium carbonate, PW 18; talc, PW 26, and barium sulfate, PW 21);
black pigments (manganese black, spinel black and carbon blacks (graphite carbon));
luster pigments (absorption pigments, metal pigments or metallic effect pigments and pearlescent pigments) and
inorganic colored pigments (iron oxide pigments, iron blue pigments, ultramarine pigments and, less suitable because of their toxicological properties, lead chromate pigments, chromium oxide pigments, cadmium pigments and bismuth vanadate pigments).

One group of particularly preferred pigments form the coloring synthetic iron oxides. Particularly preferred representatives of this substance class are Pigment Brown 6 (CI No 77491), Pigment Red 101 (CI No 77491), Pigment Yellow 42 (CI No 77492), Pigment Black 11 (CI No 77499) and mixtures of these pigments.

Very particularly preferred cosmic compositions contain starch particles, which in turn contain as pigment a synthetic inorganic pigment, preferably a synthetic inorganic pigment from the group of metal oxides and in particular a synthetic inorganic pigment from the group of iron oxides.

The starch particles contained in the cosmetic compositions preferably have a core of starch and a pigment coating at least partially surrounding this core. The starch particles are preferably prepared by coating starch with the pigment.

In addition to the starch and the pigment, the starch particles can comprise further constituents, for example, care substances. These can in particular comprise a polysiloxane, preferably a polydimethylsiloxane (dimethicone) and/or a cationic surfactant, preferably a tetraalkylammonium compound having a C₁₀-C₂₂ alkyl group such as an N,N,N-trimethylhexadecylammonium halide compound. The total amount of these further ingredients is preferably up to about 1.5% by weight and more preferably up to about 1.2% by weight, based on the total weight of the cosmetic composition.

A second essential ingredient of the cosmetic compositions is a firming compound.

Styling agents must meet a whole series of other requirements in addition to a high degree of hold. These can be roughly subdivided into properties on the hair, properties of the particular formulation, for example, properties of the foam, the gel or the sprayed paint, and properties relating to the handling of the styling agent, wherein the properties on the hair are accorded particular importance. Particularly noteworthy are moisture resistance, low tack and a balanced conditioning effect. Furthermore, a styling agent should be universally applicable for all hair types and mild to hair and skin.

A variety of synthetic polymers, which are used in styling agents, have already been developed as firming compounds to meet the different requirements. Alternatively or additionally, waxes are used as firming compounds. Ideally, the polymers and/or waxes, when applied to the hair, result in a polymer film or film which, on the one hand, gives the hairstyle a strong hold, but on the other hand, is sufficiently flexible not to break under stress.

The synthetic polymers can be subdivided into cationic, anionic, nonionic and amphoteric forming polymers.

Suitable synthetic polymers comprise, for example, polymers having the following INCI names: Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVPNA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Also suitable are cellulose ethers, such as hydroxypropylcellulose, hydroxyethylcellulose and methylhydroxypropylcellulose Synthetic polymers which are furthermore suitable as firming compounds are siloxanes. These siloxanes can be both water-soluble and water-insoluble. Both volatile and nonvolatile siloxanes are suitable, wherein nonvolatile siloxanes are understood to mean those compounds whose boiling point is above about 200° C. under normal pressure. Preferred siloxanes are polydialkylsiloxanes, such as, for example, polydimethylsiloxane, poly alkylarylsiloxanes, such as, polyphenylmethylsiloxane, ethoxylated polydialkylsiloxanes and polydialkylsiloxanes which contain amine and/or hydroxyl groups. Glycosidically substituted silicones are also suitable.

Homopolyacrylic acid (INCI: Carbomer), which is commercially available under the name Carbopol® in various designs, is also suitable as a firming compound.

Preferably, the firming compound comprises a vinylpyrrolidone-containing polymer. Most preferably, the firming compound comprises a polymer selected from the group including polyvinylpyrrolidone (PVP), vinylpyrrolidone-vinyl acetate copolymer (VP/VA copolymer), Vinyl Caprolactam/VP/Dimethylaminoethyl Methacrylate Copolymer (INCI), VP/DMAPA Acrylates Copolymer (INCI) and mixtures thereof.

Another preferred firming compound is octylacrylamide/acrylOctylacrylamide/Acrylates/Butylaminoethyl Methacrylate Copolymer (INCI), which is sold under the name “Amphomer®” by Akzo Nobel.

A further preferred firming compound is Acrylates/Hydroxyesters Acrylates Copolymer (INCI), which is sold under the name “Acudyne 1000 Hair Styling Polymer” by The Dow Chemical Company.

Accordingly, it is particularly preferred that the firming compound is a synthetic polymer selected from the group including polyvinylpyrrolidone (PVP), vinylpyrrolidone-vinyl acetate copolymer (VP/VA coplymer), Vinyl Caprolactam/VP/Dimethylaminoethyl Methacrylate Copolymer (INCI), VP/DMAPA Acrylates Copolymer (INCI), Octylacrylamide/Acrylates/Butylaminoethyl Methacrylate Copolymer (INCI), Acrylates/Hydroxyesters Acrylates Copolymer (INCI) and comprises mixtures thereof.

The cosmetic composition can contain, in addition to or in the alternative to a synthetic polymer, at least one natural or synthetic wax, which has a melting point above about 37° C., as a firming compound.

Solid paraffins or isoparaffins, vegetable waxes such as candelilla wax, carnauba wax, espalosa wax, Japan wax, cork wax, sugarcane wax, ouricury wax, montan wax, sunflower wax, fruit waxes and animal waxes such as, for example, beeswax and other insect waxes, spermaceti, shellac wax, wool wax and rump fat, furthermore, mineral waxes, such as, for example, ceresin and ozokerite or the petrochemical waxes, such as, for example, petrolatum, paraffin waxes, microwaxes of polyethylene or polypropylene and polyethylene glycol waxes can be used as natural or synthetic waxes. It can be advantageous to use hydrogenated or hardened waxes. Furthermore, it is also possible to use chemically modified waxes, in particular the hard waxes, for example, montan ester waxes, Sasol waxes and hydrogenated jojoba waxes.

Further suitable are the triglycerides of saturated and possibly hydroxylated C16-30 fatty acids, such as hardened triglyceride fats (hydrogenated palm oil, hydrogenated coconut oil, hydrogenated castor oil), glyceryl tribehenate or glyceryl tri-12-hydroxystearate, furthermore synthetic full esters of fatty acids and glycols (for example Syncrowach®) or polyols having 2 to 6 C atoms, fatty acid monoalkanolamides having a C12-22 acyl radical and a C2-4 alkanol radical, esters of saturated and/or unsaturated, branched and/or unbranched alkanecarboxylic acids having a chain length of 1 to 80 carbon atoms and saturated and/or unsaturated, branched and/or unbranched alcohols having a chain length of 1 to 80 C atoms, including, for example, synthetic fatty acid fatty alcohol esters, such as stearyl stearate or cetyl palmitate, esters of aromatic carboxylic acids, dicarboxylic acids or hydroxycarboxylic acids (for example, 12-hydroxystearic acid) and saturated and/or unsaturated, branched and/or unbranched alcohols having a chain length of from 1 to 80 carbon atoms, lactides of long-chain hydroxycarboxylic acids and full esters of fatty alcohols and di- and tricarboxylic acids, for example, dicetylsuccinate or dicetyl/stearyl adipate, and mixtures of these substances.

The wax components can also be selected from the group of esters of saturated, unbranched alkanecarboxylic acids having a chain length of 14 to 44 carbon atoms and saturated, unbranched alcohols having a chain length of 14 to 44 carbon atoms, provided the wax component or the entirety of the wax components are solid at room temperature. The wax components can be selected, for example, from the group of C16-36 alkyl stearates, C10-40 alkyl stearates, C2-40 alkyl isostearates, C20-40 dialkyl esters of dimer acids, C18-38 alkyl hydroxystearoyl stearates, C20-40 alkyl erucates, C30-50-alkyl beeswax and cetearyl behenate are further usable. Silicone waxes, for example stearyltrimethylsilane/stearyl alcohol, can also be advantageous. Preferred wax components are the esters of saturated monohydric C20-C60 alcohols and saturated C8-C30 monocarboxylic acids, in particular a C20-C40-alkyl stearate, which is obtainable under the name Kesterwachs® K82H from Koster Keunen Inc.

Natural, chemically modified and synthetic waxes can be used alone or in combination. The teaching as contemplated herein thus also comprises the combined use of several waxes. Furthermore, a series of wax mixtures, optionally in admixture with further additives, is commercially available. Those under the names “Spezialwachs 7686 OE” (a mixture of cetyl palmitate, beeswax, microcrystalline wax and polyethylene having a melting range of from about 73-75° C.; manufacturer: Kahl & Co), Polywax® GP 200 (a mixture of stearyl alcohol and polyethylene glycol stearate having a melting point of from about 47-51° C.; manufacturer: Croda) and “Weichceresin® FL 400” (a Vaseline/Vaseline oil/wax mixture having a melting point of from about 50-54° C.; manufacturer: Parafluid Mineralolgesellschaft) are examples of usable mixtures.

The wax is preferably selected from carnauba wax (INCI: Copernicia Cerifera Cera) beeswax (INCI: Beeswax), Petrolatum (INCI), microcrystalline wax, and especially mixtures thereof.

Preferred mixtures comprise the combination of carnauba wax (INCI: Copernicia Cerifera Cera), petrolatum and microcrystalline wax or the combination of beeswax (INCI: Beeswax) and petrolatum.

The wax or wax components should be solid at about 25° C. and should melt in the range>37° C.

The cosmetic composition preferably contains the firming compound in a total amount of from about 0.5 to about 50% by weight, preferably from about 1 to about 40% by weight, more preferably from about 1.5 to about 30% by weight, still more preferably from about 2 to about 25% by weight, based on the total weight of the cosmetic composition.

The cosmetic composition can comprise, as the firming compound, one or more synthetic polymers, one or more waxes or one or more synthetic polymers and one or more waxes. More preferably, the cosmetic composition contains, as the firming compound, one or more synthetic polymers or one or more synthetic polymers and one or more waxes.

The cosmetic composition can contain further ingredients. In particular, the cosmetic composition can contain water. Preferred cosmetic compositions contain water as a cosmetic carrier. In these embodiments, the cosmetic composition contains water as a main component. The water content of the cosmetic composition is preferably from about 5 to about 90% by weight, preferably from about 15 to about 80% by weight and more preferably from about 40 to about 75% by weight, based on the total weight of the cosmetic composition.

The cosmetic composition can contain at least one emulsifier as a further constituent. This is particularly advantageous when the cosmetic composition comprises at least one wax as a firming compound.

Suitable emulsifiers are, in principle, anionic, cationic, nonionic and ampholytic surface-active compounds which are suitable for use on the human body. The ampholytic surface active compounds comprise zwitterionic surface active compounds and ampholytes. Preference is given to nonionic emulsifiers.

In particular, addition products of ethylene oxide to linear fatty alcohols, to fatty acids, to fatty acid alkloamides, to fatty acid monoglycerides, to sorbitan fatty acid monoesters, to fatty acid glycerides, to methylglucoside monofatty acid esters, to polydimethylsiloxanes and mixtures thereof are useful as nonionic emulsifiers.

The emulsifier is preferably selected from nonionic emulsifiers such as addition products of 2 to 50 moles of ethylene oxide to linear fatty alcohols having 8 to 30, preferably 12 to 18 carbon atoms, addition products of 2 to 50 moles of ethylene oxide and 1 to 5 moles of propylene oxide to linear fatty alcohols having to 8 to 30, preferably 12 to 18 carbon atoms, addition products of 2 to 100 moles of ethylene oxide to linear fatty acids having 12 to 18 carbon atoms and mixtures thereof.

Examples of particularly preferred nonionic surfactants are compounds having the INCI names Steareth-2, Steareth-21, Oleth-10, PEG-100 Stearate or PPG-5-Ceteth-20 and in particular combinations thereof.

Emulsifiers also preferred are the esters of fatty acids having 12 to 18 carbon atoms with saccharides. In particular, the mono- and/or diesters of saccharose (sucrose) with stearic and/or palmitic acid are preferably used. Examples of such nonionic emulsifiers are compounds having the INCI names Sucrose Stearate, Sucrose Distearate and mixtures thereof.

Further preferred emulsifiers are linear fatty acids having 12 to 18 carbon atoms and mixtures thereof. The linear fatty acids can be neutralized and/or non-neutralized, depending on the pH. Preferred fatty acids comprise stearic and/or palmitic acid.

Emulsifiers also preferred are addition products of 2 to 20 moles of ethylene oxide to beeswax, in particular compounds having the INCI names PEG-6 Beeswax, PEG-8 Beeswax, PEG-12 Beeswax or PEG-20 Beeswax. PEG-8 Beeswax is particularly preferred from this class of emulsifiers.

A further class of emulsifiers which can preferably be used are the monoesters of fatty acids having 12 to 22 carbon atoms with glycerin. In particular, the monoesters of glycerin with stearic and/or palmitic acid are preferably used. Examples of particularly preferred emulsifiers are compounds having the INCI names Glyceryl Stearate, Glyceryl Palmitate or mixtures thereof.

It is particularly preferred that the emulsifier is a nonionic emulsifier selected from the group including addition products of 2 to 50 moles of ethylene oxide to linear fatty alcohols having 12 to 18 carbon atoms, addition products of 2 to 100 moles of ethylene oxide to linear fatty acids having 12 to 18 carbon atoms, linear fatty acids having 12 to 18 carbon atoms and comprises mixtures thereof.

The cosmetic composition preferably contains the emulsifier in a total amount of from about 0.1 to about 30% by weight, preferably from about 0.5 to about 20% by weight, more preferably from about 1 to about 15% by weight, based on the total weight of the cosmetic composition.

Further suitable auxiliaries and additives are, in particular, care components, such as oils, protein hydrolyzates and/or derivatives thereof, vitamins, provitamins, vitamin precursors and/or derivatives thereof or linear fatty alcohols having 12 to 18 carbon atoms.

The cosmetic agent can further contain neutralizers or pH adjusters for adjusting the pH.

The compositions can further contain cosmetically acceptable preservatives, UV filters and/or perfumes. It can be advantageous for the compositions to contain a solubilizer such as PEG-40 Hydrogenated Castor Oil to stabilize a perfume in the compositions.

It is preferred for the cosmetic composition to be free of hydrophobically modified SiO₂, in particular free of hydrophobically modified silicic acid.

The term “free of a compound” in the context of this application means that the cosmetic composition contains no or only extremely small amounts (maximum up to about 0.2% by weight of the total amount of cosmetic composition) of the compound concerned. It is very particularly preferred for the cosmetic composition to contain no hydrophobically modified SiO₂.

The cosmetic composition will be formulated in the forms customary for the temporary deformation of keratinic fibers, for example, as wax, paste, cream, lotion or clay. The product form “clay” refers to highly viscous, waxy cosmetic agents that contain, among other things, clay compounds (for example kaolin). The cosmetic compositions are preferably offered in tubes, cans or pots.

The cosmetic composition is preferably propellant-free. Accordingly, the cosmetic composition is not an aerosol. Propellants in the context of this application are liquefied or compressed gases such as dimethyl ether, chlorofluorocarbons (CFCs), carbon dioxide, hydrocarbons, air and nitrogen.

The present disclosure also relates to the use of a cosmetic composition containing, based on its total weight
a) from about 1 to about 10% by weight of starch particles comprising, based on the weight of the starch particles,
a1) from about 70 to about 99% by weight of starch and
a2) from about 1 to about 30% by weight of pigment,
b) from about 0.5 to about 50% by weight of a firming compound selected from the group including waxes, synthetic polymers and mixtures thereof.
   for temporary deformation of keratinic fibers. For this purpose, the cosmetic composition is applied to these before or after the deformation of the keratinic fibers. Hands, in particular fingers, or a comb can be used for application of the cosmetic composition.

The composition of some preferred cosmetic compositions can be found in the following table (specifications as active substance content and in % by weight relative to the total weight of the cosmetic composition, unless stated otherwise).

| Component/INCI name: | Example 1 | Example 2 |
|---|---|---|
| Beeswax | 2-6 | — |
| Petrolatum | 2-8 | — |
| Cetearyl Alcohol | 1-3 | — |
| Palmitic Acid | 2-5 | — |
| Stearic Acid | 2-5 | — |
| Cetyl Alcohol | 2-5 | — |
| C12-15 alkyl benzoates | 4-8 | — |
| Benzophenone-4 | — | 0.05-0.5 |
| PVP | 0.5-4 | 0.5-4 |
| VP/VA Copolymer | — | 1-5 |
| Carbomer | — | 0.1-0.8 |
| PPG-5-ceteth-20 | 0.5-3 | — |
| Oleth-20 | 0.5-3 | — |
| Steareth-21 | 0.5-3 | — |
| Steareth-2 | 0.5-3 | — |
| Paraffinum Liquidum | 0.5-3 | — |
| Aminomethyl propanol | — | 0.1-0.6 |
| Oryza Sativa (Rice) Starch | 2-5 | 4-8 |
| CI 77499 (Iron Oxides) | 0.2-2 | 0.5-2.5 |
| CI 77491 (Iron Oxides) | — | 0.05-0.5 |
| CI 77492 (Iron Oxides) | — | 0.05-0.5 |
| Cetrimmonium Chloride | 0-0.2 | 0-0.2 |
| Dimethicone | 0.2-1 | 0.01-0.5 |
| PEG-40 Hydrogenated Castor Oil | — | 0-1 |
| Panthenol | — | 0.05-0.5 |
| Preservative | 0-1.5 | 0-1.5 |
| Perfume (fragrance) | 0-1.5 | 0-1.5 |
| Aqua (Water) | ad 100 | ad 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A cosmetic composition for the temporary reshaping of keratinic fibers, comprising, based on its total weight
a) from about 1 to about 10% by weight of starch particles comprising, based on the weight of the starch particles,
a1) from about 70 to about 99% by weight of starch, and
a2) from about 1 to about 30% by weight of pigment,
b) from about 0.5 to about 50% by weight of a firming compound selected from the group comprising waxes, synthetic polymers and mixtures thereof.

2. The cosmetic composition according to claim 1, wherein
the starch particles have a coating of the pigment.

3. The cosmetic composition according to claim 17, wherein
the pigment is selected from the group comprising Pigment Brown 6 (CI No 77491), Pigment Red 101 (CI No 77491), Pigment Yellow 42 (CI No 77492), Pigment Black 11 (CI No 77499) and mixtures of these pigments.

4. The cosmetic composition according to claim 1, wherein
the starch particles comprise a further constituent selected from the group comprising polydimethylsiloxanes, cationic surfactants and mixtures thereof.

5. The cosmetic composition according to claim 1, wherein
the firming compound comprises a wax selected from the group comprising vegetable waxes, Petrolatum (INCI), beeswax (INCI: Beewax), microcrystalline waxes and mixtures thereof.

6. The cosmetic composition according to claim 1, wherein
the firming compound comprises a synthetic polymer selected from the group comprising polyvinylpyrrolidone (PVP), vinyl pyrrolidone-vinyl acetate copolymer (VP/VA coplymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI) and mixtures thereof.

7. The cosmetic composition according to claim 1, wherein
the cosmetic composition is free of propellant.

8. The cosmetic composition according to claim 1, wherein
the cosmetic composition is free of hydrophobically modified SiO₂.

9. The cosmetic composition according to claim 1 further comprising an emulsifier.

10. The cosmetic composition according to claim 9, wherein the emulsifier comprises a nonionic emulsifier.

11. The cosmetic composition according to claim 1, wherein the starch is selected from the group comprising tapioca starch, potato starch, corn starch, rice starch, and mixtures thereof.

12. The cosmetic composition according to claim 1, wherein the starch particles have an average particle diameter of from about 5 to about 100 μm.

13. The cosmetic composition according to claim 1, wherein the starch particles are present in the cosmetic composition in an amount of from about 1.5 to about 8% by weight of the total weight of the cosmetic composition.

14. The cosmetic composition according to claim 1, wherein the starch particles comprise, based on the total weight of the starch particles, from about 1 to about 30% by weight of pigment.

15. The cosmetic composition according to claim 1, wherein the starch particles comprise, based on the total weight of the starch particles, from about 75 to about 96% by weight of starch.

16. The cosmetic composition according to claim 1, wherein the pigment is selected from the group comprising mineral pigments, metal pigments, and pearlescent pigments.

17. The cosmetic composition according to claim 4, wherein the constituent is present in the starch particle in an amount of up to about 1.5% by weight based on the total weight of the cosmetic composition.

18. The cosmetic composition according to claim 5, wherein the wax comprises a combination of carnauba wax (INCI: Copernicia Cerifera Cera), petrolatum and microcrystalline wax or a combination of beeswax (INCI: Beeswax) and petrolatum.

19. A cosmetic composition having dyeing properties comprising:
a) starch particles comprising a starch core coated with a pigment coating; and
b) a firming compound selected from the group comprising waxes, synthetic polymers and mixtures thereof.

20. The cosmetic composition according to claim 20, wherein the starch core comprises starch selected from the group comprising tapioca starch, potato starch, corn starch, rice starch, and mixtures thereof and the pigment coating comprises a pigment selected from the group comprising mineral pigments, metal pigments, and pearlescent pigments.
